(19) United States
(12) Patent Application Publication
(10) Pub. No.: US 2018/0110463 A1
(43) Pub. Date: Apr. 26, 2018

(54) BIO-IMPEDENCE-BASED SLEEP BREATHING STATE SIGNAL ACQUISITION DEVICE AND MONITORING SYSTEM

(71) Applicant: SEALAND TECHNOLOGY (CHENGDU) LIMITED, Chengdu (CN)

(72) Inventors: Tao Dai, Chengdu (CN); Xianhong Xu, Chengdu (CN); Song Gao, Chengdu (CN); Yigang Wang, Chengdu (CN)

(21) Appl. No.: 15/565,886

(22) PCT Filed: May 10, 2016
(86) PCT No.: PCT/CN2016/081480

§ 371 Date: Oct. 11, 2017

(30) Foreign Application Priority Data
May 4, 2015 (CN) 201510220522.7

(51) Int. Cl. A61B 5/00; A61B 5/0205

(52) U.S. Cl. CPC A61B 5/4818; A61B 5/6843; A61B 5/0809; A61B 5/6838; A61B 5/0205; A61B 5/0004

(57) ABSTRACT

A signal acquisition device (100) of sleep breathing state based on bio-impedance and a monitoring system are provided. The signal acquisition device (100) includes a signal acquisition unit (101), a processing control unit (102), a data storage unit (103) and a wireless communication unit (104). The signal acquisition unit (101) includes electrodes (1011, 1012), a chest impedance acquisition module (1013) connected with the electrodes (1011, 1012), a heart rate acquisition module (1014) connected with the electrodes, and a snore acquisition module (1015). The signal acquisition unit (101) can collect signal data through three channels synchronously, chest impedances and heart rate signals are acquired by using bio-impedance technology, and snore audio signals are combined to monitor sleep breathing state. Furthermore, wireless signal transmission is used to prevent directly connecting with the patient via wires, the device is simple and cheap, convenient to wear, and applicable to domestic utilization.

### FIELD OF THE INVENTION

The present invention relates to technical field of biomedical monitoring, and more particularly to a signal acquisition device of sleep breathing state based on bio-impedance, and a monitoring system with the same.

### BACKGROUND OF THE INVENTION

Bio-impedance technology is noninvasive detection technology by using electrical characteristics of biological tissues and organs to fetch physiological information or pathological information for human body. Different human tissues and organs have different bio-impedance characteristics, and the bio-impedance characteristics may be varied as the state changes or function changes of the tissues and organs. For example, in the prior art, diaphragm fatigue is divided into different levels, according to the synchronization of the diaphragm fatigue and the peaks of the chest respiratory electrical impedance signal and the abdominal respiratory electrical impedance signal. Bio-impedance technology has great potential and value in fields of clinical medicine or health care, since it's non-invasive and low-cost, and easy to carry out a long-term and real-time monitoring.

The monitoring of sleep breathing state has become a focus in the sleep medicine, since such a monitoring is directly related to sleep disorders. In sleep disorders, sleep apnea is one type of dyssomnias with respiratory arrest occurs during the sleep, specifically, this apnea is a clinical syndrome of chronic hypoxemia and hypercapnia which is resulted by apnea occurred over 30 times in a continuous 7-hour sleep for 10s or longer for each time; or apnea hyponea index is more than 5 times. Specifically, apnea can be divided into central type, obstructive type and mixed type. Therein, obstructive sleep apnea-hypopnea syndrome (OSAHS) is defined as apnea caused by soft tissue relaxation near the throat to bring upper respiratory tract stenosis or even obstruction during the sleep. Central sleep apnea-hypopnea syndrome (CSAHS) is one respiratory disorder caused by central nervous system function disorders, instead of airway obstruction; usually time of airflow through the upper airway is no more than 10 seconds, without thoracoabdominal breathing. While the mixed type means the mixture of the mentioned two types. At present, according to statistics, the number of people with respiratory sleep disorders is accounted for 2% to 4% of the total population, and with a significant increasing trend. Clinical syndrome with a series of pathophysiological changes caused by apnea/hypopnea, hypercapnia, and sleep disruption in the sleep is known as sleep apnea hypopnea syndrome (SAHS). While early diagnosis and treatment for the above-mentioned sleep apnea may improve the quality of life of patients and prevent various complications. Therefore, the monitoring of sleep breathing is the primary step in the prevention and treatment for the sleep apnea.

There are a variety of sleep breathing monitoring methods that have been used clinically. For example, polysomnography (PSG) sleep monitor, which is the gold standard for the diagnosis of sleep apnea hypopnea syndrome. PSG monitor can record the sleep through electroencephalography, eye movements, and electromyography, analyze the sleep, and monitor the patient's breathing, body movements and blood pressure. Therefore, the advent of PSG on the study of the sleep has a decisive significance. The PSG-based sleep breathing detection technology can accurately detect the abnormal breathing phenomenon, but this detecting means requires patients to wear masks, thoracoabdominal belts and multiple electrodes, thus bring a great physiological load and mental load for the patients to be prone to “First night effect” which may affect the results of the sleep monitoring. Moreover, the operation is complex and the detection is expensive. Meanwhile, since there are a little current professional diagnosis organizations for sleep disorders, thus patients must queue up for a long time to accept an inspection; further, a failed experiment failed or an experimental result deviation will be generated because the patients in a strange environment and with multiple wires connected to the body are not easy to fall asleep or have light sleep, and easy to wake up at night. In addition, in order to help doctors to provide diagnostic references or treatment programs, and facilitate their own initial screening, how to effectively monitor the sleep state for patients with sleep apnea has become a new market demand.

### SUMMARY OF THE INVENTION

To solve the technical problems mentioned above, the objective of the present invention is to provide a signal acquisition device of sleep breathing state and a monitoring system that are simple and easy-to-use and are suitable to individuals and families.

To solve the technical problems mentioned above, the present invention provides a signal acquisition device of sleep breathing state based on bio-impedance, which includes:

a signal acquisition unit, comprising electrodes to be in contact with a human chest, a chest impedance acquisition module connected with the electrodes, a heart rate acquisition module connected with the electrodes, and a snore acquisition module;

a processing control unit, connected with the chest impedance acquisition module, the heart rate acquisition module and the snore acquisition module respectively, and arranged for converting an acquisition signal to digital signal data and controlling to send out a notice or an alarm to a detected object under a normal or abnormal condition;

a data storage unit, connected with the processing control unit and arranged for storing the digital signal data; and

a wireless communication unit, connected with the data storage unit and arranged for transmitting the digital signal data to an external terminal.

As an embodiment, the device further includes:

a LED, connected with the processing control unit and arranged for indicating working status and power status of the signal acquisition device;

an output interface, connected with the data storage unit and arranged for transmitting acquisition data;

a housing, arranged for accommodating and protecting the signal acquisition unit, the processing control unit, the data storage unit, the wireless communication unit and a power supplying unit; and

a plug port, connected with the chest impedance acquisition module and the heart rate acquisition module respectively.

As another embodiment, the device further includes a clamp which is configured on an outer surface of a housing and arranged for fixing the human body to the signal acquisition device.

Preferably, the electrodes are exciting electrode or collecting electrodes.

As an embodiment, the output interface and the plug port are located at the same surface of the housing.

As an embodiment, the electrodes are mounted on an electrode connector at one end of a wire, and a plug port of the chest impedance acquisition module and the heart rate acquisition module is connected to a plug connector at another end of the wire.

As an embodiment, the electrodes and a wire are connected to form an integrated lead wire which is connected with a plug port of the chest impedance acquisition module and the heart rate acquisition module.

As an embodiment, the snore acquisition module has an acquisition front which is a microphone, and the microphone is located at an outer surface of the housing.

As an embodiment, the signal acquisition device has a low battery and is required to be charged or replaced with a new battery, once the LED is indicated to be red; the signal acquisition device has a sufficient battery and is located in a normal state, once the LED is indicated to be green; while the signal acquisition device is suggested to generate a poor contact between the electrodes and the human body and required to adjust contact positions between the electrodes and the human body, once the LED is indicated to be yellow.

A monitoring system with the signal acquisition device of sleep breathing state based on bio-impedance is provided, which further includes a terminal processing device wirelessly connected with the signal acquisition device.

As an embodiment, the terminal processing device includes:

a data analysis module, wirelessly connected with the signal acquisition device and arranged for receiving acquisition digital data, performing matching analysis of chest impedance data and heart rate data in the acquisition digital data, and obtaining snore data as reference data of sleep state;

a real-time data display module, connected with the data analysis module to display changing curves for chest impedance, heart rate and snore; and

a storage and replay module, connected with the real-time data display module and arranged for storing processing results of the acquisition data, thereby facilitating to perform history view.

In comparison of the prior art, in the present invention, chest impedances and heart rate signals are acquired by using bio-impedance technology, and snore audio signals are combined to monitor sleep breathing state; furthermore, wireless signal transmission is used, therefore problems that a patient difficultly falls asleep, easily wakes up at night, or is in a light sleep, caused by the fact that his body is connected with a lead wire, are avoided. The monitoring system of sleep breathing state is simple and cheap, convenient to wear, and applicable to domestic utilization, which may not affect sleep; moreover, the monitoring system is used in daily real sleep environment, thus a real-time sleep breathing state monitoring during patient's real sleep can be done without influence of sleep posture change.

### BRIEF DESCRIPTION OF THE DRAWINGS

FIG. 1 is a schematic view of a signal acquisition device of sleep breathing state according to a first embodiment of the present invention;

FIG. 2 is a plane view of the signal acquisition device of sleep breathing state according to the first embodiment of the present invention;

FIG. 3 is a side view of the signal acquisition device of sleep breathing state according to the first embodiment of the present invention;

FIG. 4 is a schematic view of lead wires of the signal acquisition device of sleep breathing state according to the first embodiment of the present invention;

FIG. 5 is a top view of the signal acquisition device of sleep breathing state, without lead wires connected according to the first embodiment of the present invention;

FIG. 6 is a schematic view showing a monitoring system of sleep breath state connected with human body according to a second embodiment of the present invention;

FIG. 7 is a schematic view of the monitoring system of sleep breath state according to the second embodiment of the present invention;

FIG. 8 is a curve of chest impedance (breathing) change detected by the monitoring system of sleep breath state according to the second embodiment of the present invention;

FIG. 9 is a curve of heart rate change detected by the monitoring system of sleep breath state according to the second embodiment of the present invention;

FIG. 10 is a decibel curve of snore detected by the monitoring system of sleep breath state according to the second embodiment of the present invention.

### DETAILED DESCRIPTION OF ILLUSTRATED EMBODIMENTS

The signal acquisition device of sleep breathing state and the monitoring system thereof according to the present invention use bio-impedance technology to obtain sleep respire monitoring signals, and take snore signals as reference, thereby detecting apnea time, diagnosis of apnea type and sleep quality during the sleep, which is especially applicable to sub-health people to do self check. Following is the preferred embodiments of the present invention, which will not limit the scope of the present invention however.

### Embodiment 1

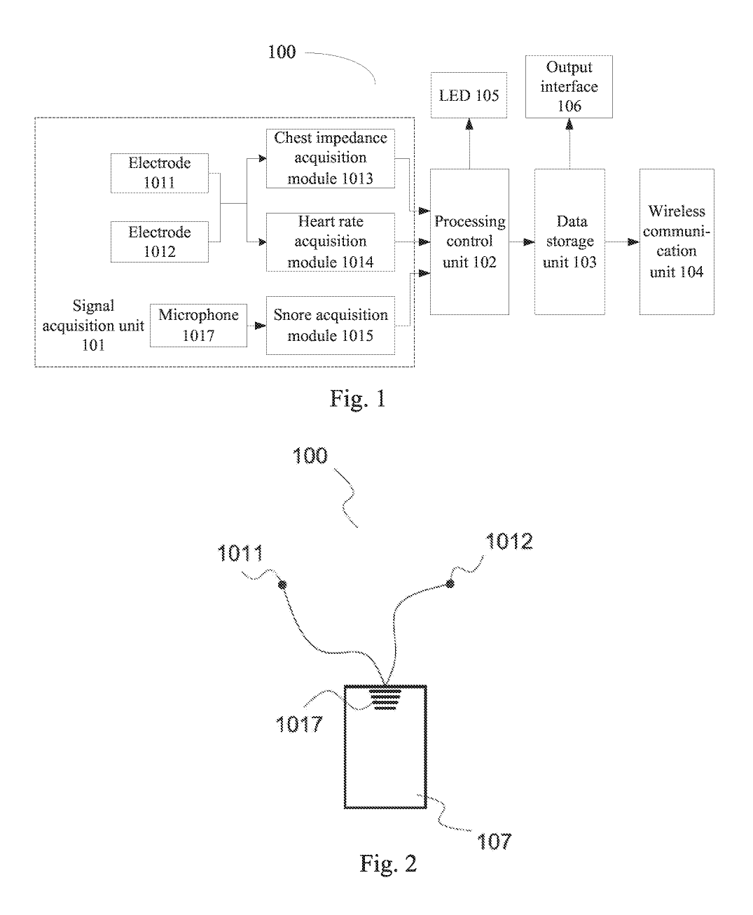

As shown in FIG. 1, the present invention provides a signal acquisition device 100 of sleep breathing state based on bio-impedance, the device 100 including a signal acquisition unit 101, a processing control unit 102, a data storage unit 103 and a wireless communication unit 104.

Specifically, the signal acquisition unit 101 includes at least two electrodes 1011 and 1012 to be in contact with a human chest, a chest impedance acquisition module 1013 connected with the electrode 1011 or 1012, a heart rate acquisition module 1014 connected with the electrodes 1011 or 1012, and a snore acquisition module 1015. In this embodiment, both of the electrodes 1011 and 1012 are exciting electrode or collecting electrodes, dual electrodes are used to perform excitation and collection as the same time. The processing control unit 102 is connected with the chest impedance acquisition module 1013, the heart rate acquisition module 1014 and the snore acquisition module 1015 respectively, and arranged for converting acquisition signals to digital signal data. The data storage unit 103 is connected with the processing control unit 102 and arranged for storing the digital signal data from the processing control unit. The wireless communication unit 104 is connected with the data storage unit 103 and arranged for transmitting the digital signal data to an external terminal. Every unit mentioned above is provided with a power unit (not shown) to supply power, such as external power or internal battery, and preferably a power switch can be configured to connect with the power unit.

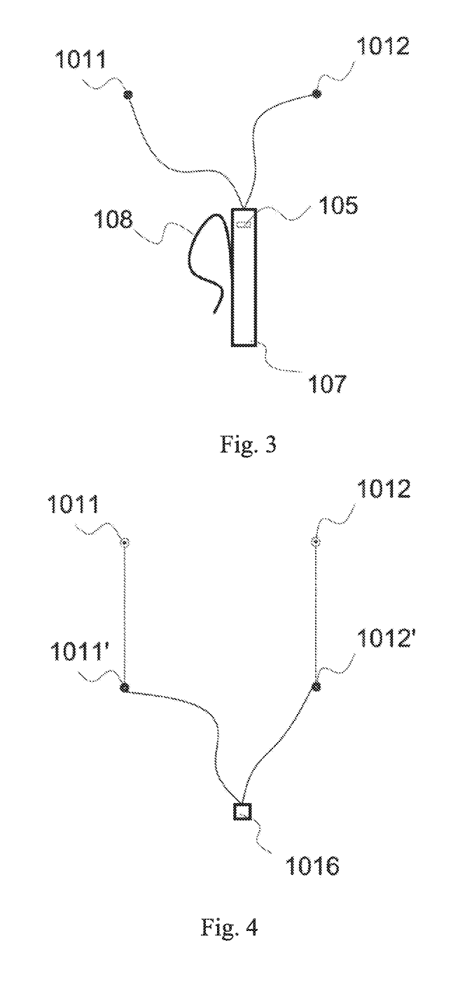

As shown in FIGS. 2 and 3, the signal acquisition device 100 further includes a LED 105, an output interface 106, a housing 107 and a plug port 109.

Specifically, the LED 105 is connected with the processing control unit 102 and arranged for indicating working status and power status of the signal acquisition device 100. If the LED 105 is indicated to be red, that means the power or battery of the signal acquisition device 100 is in a low level and it should be charged or replaced with a new battery; if the LED 105 is indicated to be green, that means the signal acquisition device 100 has sufficient power or battery and is under normal condition to be used; if the LED 105 is indicated to be yellow, that means poor contact between the electrodes of the signal acquisition device 100 and the human body occurs, which advises user to adjust the contact position between the electrode and the human body.

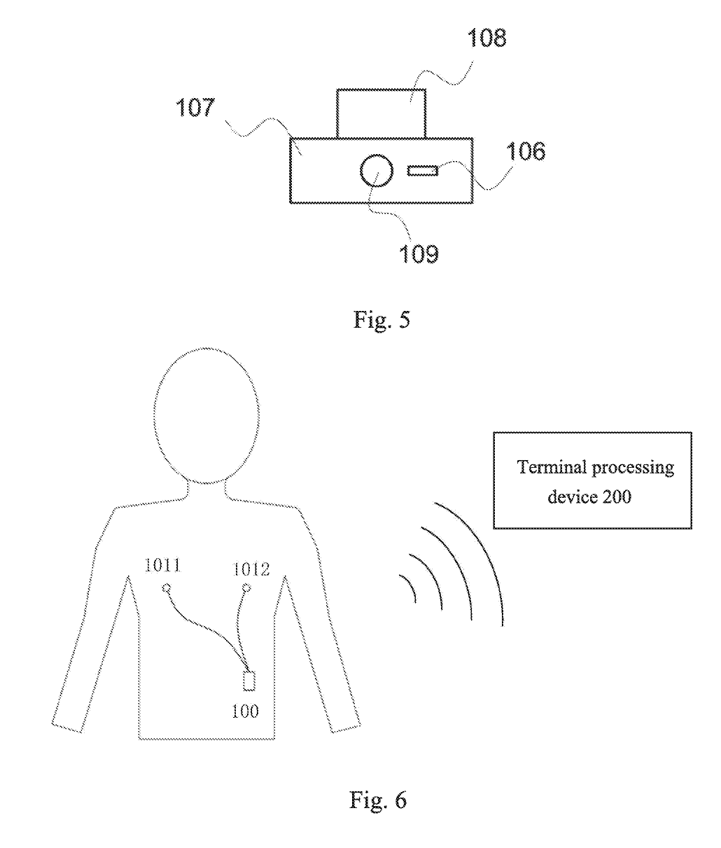

In addition, the output interface 106 is connected with the data storage unit 103 and arranged for outputting the acquisition data, so that the doctors could be aware of the usual sleep mode of the patients. The housing 107 is arranged for accommodating and protecting the signal acquisition unit 101, the processing control unit 102, the data storage unit 103 and the wireless communication unit 104. In this embodiment, the protecting the signal acquisition unit 101, the processing control unit 102, the data storage unit 103 and the wireless communication unit 104 are integrated in the circuit board, and the plug port 109 is connected with the chest impedance acquisition module 1013 and the heart rate acquisition module 1014 respectively. As shown in FIG. 5, for reducing the mutual influence of collecting and charging, the output interface 106 and the plug port 109 are located at the same surface of the housing 107 preferably.

As shown in FIG. 3, the signal acquisition device 100 further includes a clamp 108 which is configured on an outer surface of the housing 107 and arranged for fixing the signal acquisition device 100 to the human body.

As shown in FIG. 4, the electrode 1011 (or 1012) is mounted on the electrode connector 1011′ (or 1012′) at one end of the wire, and the plug interface 1016 is provided at another end of the wire to connect with the plug port 109 of the chest impedance acquisition module 1013 and the heart rate acquisition module 1014. Further, the electrode 1011 (or 1012) is connected with the wire to form integrated lead wire which is to be connected with the plug port 109 of the chest impedance acquisition module 1013 and the heart rate acquisition module 1014. Specifically, an acquisition front the snore acquisition module 1015 is a microphone 1017 which is located at an outer surface of the housing 107.

### Embodiment 2

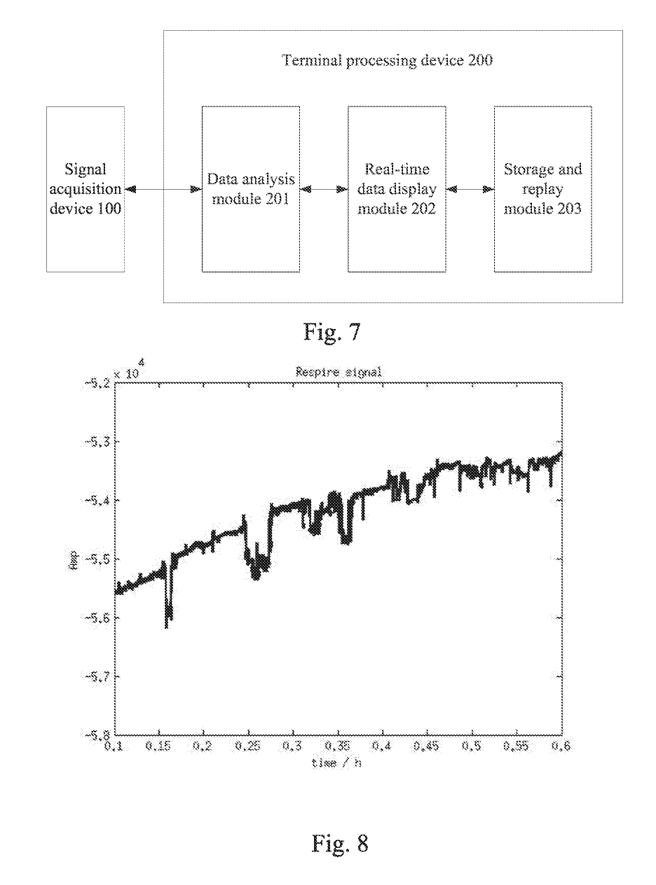

As shown in FIGS. 6 and 7, a monitoring system including the above-mention signal acquisition device 100 is provided, which further includes a terminal processing device 200 wirelessly connected to the signal acquisition device 100. In this embodiment, the terminal processing device 200 includes a data analysis module 201, a real-time data display module 202 and a storage and replay module 203.

Specifically, the data analysis module 201 is wirelessly connected with the signal acquisition device 100 for receiving the acquisition digital data, performing matching analysis of the chest impedance data and the heart rate data in the acquisition digital data, and obtaining snore data as reference data of sleep state. The real-time data display module 202 is connected with the data analysis module 201 to display the changing curves for the chest impedance, the heart rate and the snore. Further, the storage and replay module 203 is connected to the real-time data display module 202 to store the processing results of the acquisition data, thereby facilitating to view history.

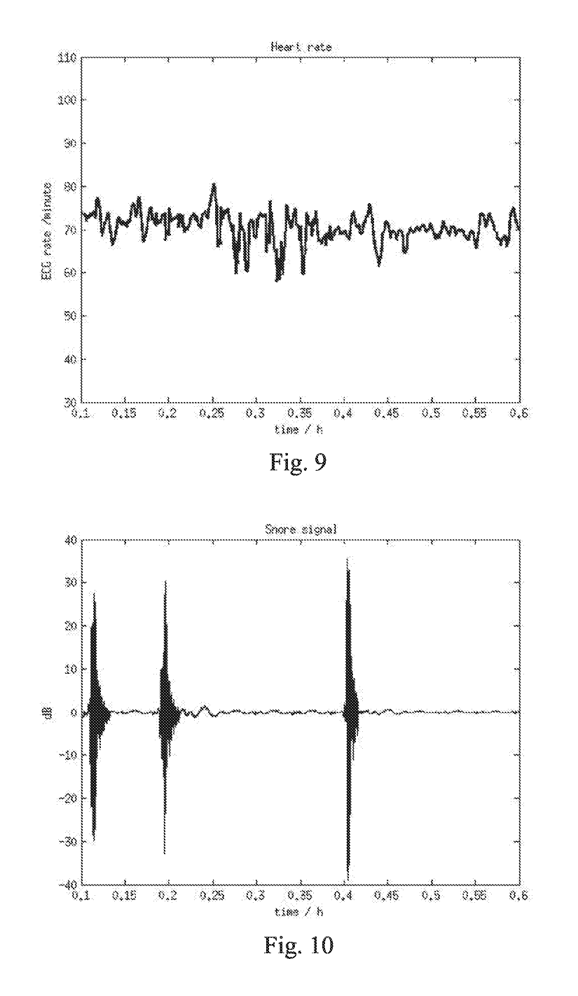

Comparing with the prior art which uses single electrode mode to collect respire signals from ECG signals, the present invention uses three channels that are chest impedance acquisition module, heart rate acquisition module and snore acquisition module to individually collect signal data of breathing impedance, heart rate and snore, as shown in FIGS. 8-10. Such a monitoring system can provide important reference information for detecting OSAHS and diagnosing different types for OSAHS.

The above descriptions are considered to be the preferred embodiments, it is to be understood that the invention is not to be limited to the disclosed embodiments, but on the contrary, is intended to cover various modifications and equivalent arrangements included within the spirit and scope of the invention.

1. A signal acquisition device of sleep breathing state based on bio-impedance, comprising:
a signal acquisition unit, comprising electrodes to be in contact with a human chest, a chest impedance acquisition module connected with the electrodes, a heart rate acquisition module connected with the electrodes, and a snore acquisition module;
a processing control unit, connected with the chest impedance acquisition module, the heart rate acquisition module and the snore acquisition module respectively, and arranged for converting an acquisition signal to digital signal data and controlling to send out a notice or an alarm to a detected object under a normal or abnormal condition;
a data storage unit, connected with the processing control unit and arranged for storing the digital signal data; and
a wireless communication unit, connected with the data storage unit and arranged for transmitting the digital signal data to an external terminal.

2. The signal acquisition device of sleep breathing state based on bio-impedance according to claim 1, further comprising:
a LED, connected with the processing control unit and arranged for indicating working status and power status of the signal acquisition device;
an output interface, connected with the data storage unit and arranged for transmitting acquisition data;
a housing, arranged for accommodating and protecting the signal acquisition unit, the processing control unit, the data storage unit, the wireless communication unit and a power supplying unit; and
a plug port, connected with the chest impedance acquisition module and the heart rate acquisition module respectively.

3. The signal acquisition device of sleep breathing state based on bio-impedance according to claim 1, further comprising a clamp which is configured on an outer surface of a housing and arranged for fixing the human body to the signal acquisition device.

4. The signal acquisition device of sleep breathing state based on bio-impedance according to claim 2, further comprising a clamp which is configured on an outer surface of a housing and arranged for fixing the signal acquisition device to the human body.

5. The signal acquisition device of sleep breathing state based on bio-impedance according to claim 1, wherein the electrodes are exciting electrode or collecting electrodes.

6. The signal acquisition device of sleep breathing state based on bio-impedance according to claim 2, wherein the output interface and the plug port are located at the same surface of the housing.

7. The signal acquisition device of sleep breathing state based on bio-impedance according to claim 1, wherein the electrodes are mounted on an electrode connector at one end of a wire, and a plug port of the chest impedance acquisition module and the heart rate acquisition module is connected to a plug connector at another end of the wire.

8. The signal acquisition device of sleep breathing state based on bio-impedance according to claim 1, wherein the electrodes and a wire are connected to form an integrated lead wire which is connected with a plug port of the chest impedance acquisition module and the heart rate acquisition module.

9. The signal acquisition device of sleep breathing state based on bio-impedance according to claim 1, wherein the snore acquisition module has an acquisition front which is a microphone, and the microphone is located at an outer surface of the housing.

10. The signal acquisition device of sleep breathing state based on bio-impedance according to claim 2, wherein the signal acquisition device has a low battery and is required to be charged or replaced with a new battery, once the LED is indicated to be red; the signal acquisition device has a sufficient battery and is located in a normal state, once the LED is indicated to be green; while the signal acquisition device is suggested to generate a poor contact between the electrodes and the human body and required to adjust contact positions between the electrodes and the human body, once the LED is indicated to be yellow.

11. A monitoring system with the signal acquisition device of sleep breathing state based on bio-impedance according to claim 1, comprising a terminal processing device wirelessly connected with the signal acquisition device.

12. The monitoring system according to claim 11, wherein the terminal processing device comprises:
a data analysis module, wirelessly connected with the signal acquisition device and arranged for receiving acquisition digital data, performing matching analysis of chest impedance data and heart rate data in the acquisition digital data, and obtaining snore data as reference data of sleep state;
a real-time data display module, connected with the data analysis module to display changing curves for chest impedance, heart rate and snore; and
a storage and replay module, connected with the real-time data display module and arranged for storing processing results of the acquisition data, thereby facilitating to perform history view.
